(19) United States
(12) Patent Application Publication
(10) Pub. No.: US 2004/0180102 A1
(43) Pub. Date: Sep. 16, 2004

(54) Compositions containing peptide copper complexes and phytochemical compounds, and methods related thereto

(75) Inventor: Leonard M. Patt, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: ProCyte Corporation, Redmond, WA

(21) Appl. No.: 10/627,193

(22) Filed: Jul. 24, 2003

Related U.S. Application Data
(60) Provisional application No. 60/400,318, filed on Jul. 31, 2002.

(51) Int. Cl. A61K 35/78; A61K 38/16; A61K 31/353

(52) U.S. Cl. 424/729; 514/6; 424/776; 514/456

(57) ABSTRACT

Novel compositions having anti-oxidant, anti-inflammatory and/or cosmetic utility for a mammal, combine at least one peptide copper complex and at least one phytochemical compound. More particularly, disclosed are such novel compositions where the phytochemical compound is a polyphenol or a carotenoid, the polyphenol being a flavanoid, a flavonoid, a flavonoid derivative, a flavolignan, a polyphenolic rhizome, or a mixture thereof. Compositions for topical application are disclosed that include additives such as emollients, sunscreen agents, skin protectants, skin conditioning agents, and humectants. Also disclosed are methods, employing such compositions, for enhancing or restoring the resistance of a mammal to oxidative or inflammatory damage, for accelerating wound healing, for cosmetically healing mammalian skin, and for stimulating hair growth, or preventing or treating hair loss.

### CROSS-REFERENCE TO RELATED APPLICATION

This application claims the benefit of U.S. Provisional Patent Application No. 60/400,318 filed Jul. 31, 2002, which application is incorporated herein by reference in its entirety.

### BACKGROUND OF THE INVENTION

1. Field of the Invention

The present invention generally relates to compositions comprising copper peptide complexes and phytochemical compounds and, more particularly, to pharmaceutical preparations and cosmetic preparations for skin comprising such compositions, as well as to methods that use such preparations for treating or preventing various conditions and diseases, including those related to oxidative or inflammatory processes.

2. Description of the Related Art

Today, more than ever, we are exposed to environmental triggers that stimulate the production of oxidants, in particular, free radicals. An exemplary free radical is the superoxide anion, O₂⁻, which is an unstable oxygen molecule created by, as examples, cigarette smoke, air pollution, ultraviolet light, pesticides, radiation, emotional stress, and excessive exercise. Such oxygen radicals, and the oxidative stress associated therewith, have been implicated in, for example, autoimmune diseases; arthritis; tissue damage related to environmental factors, as well as to surgery and transplantation; cancer; blockages in arteries; and a variety of other conditions. As another example, the oxidative stress resulting from exposure to free radical-producing triggers can accelerate the aging process of the skin, manifested, for example, in the premature appearance of wrinkles and sunspots.

Free radicals are generally ubiquitous, and the body is naturally endowed with endogenous anti-oxidants that serve to scavenge free radicals. However, it is beneficial to use anti-oxidants in and on the body in supplemental form, as the quantity of the endogenous variety may be inadequate. Thus, supplemental use of anti-oxidants is important in, to mention only a few examples, preventing disease such as, skin cancer, maintaining youthful and healthy skin, and promoting wound healing. The effectiveness of such supplemental anti-oxidants stems from their ability to scavenge free radicals and/or inhibit free radical-generating enzymes.

Oxidative stress and inflammatory conditions are closely related. In fact, inflammation has been described as both a free radical-generated and a free radical-producing process. Various sources of injury to tissue in the human body can instigate an inflammatory response. Such injury may be caused by, for example, toxins secreted by bacteria and viruses, excessive heat or cold, mechanical intrusions (i.e, tissue being cut or crushed by some object), excessive exposure to acids or alkalis, or to irradiation, or exercise-induced muscle damage. Injured cells and surrounding vascular cells excrete free radical-generating enzymes and cytokines. The excretion of the latter results in a very brief constriction of the surrounding arterioles (ischemia), followed by reperfusion, which, in turn, generates considerable reactive oxygen and free radicals, in turn, causing increased oxidative stress. Also, accumulation of fluid at the site of the injury causes swelling, or edema.

Thus, anti-oxidants tend to also function as anti-inflammatory agents in neutralizing the pro-inflammatory effect of free radicals, either through scavenging the free-radicals or inhibiting free-radical generating enzymes. Inflammatory and inflammatory-related conditions include: exercise-induced muscle damage, arthritis, peptic ulcer disease, eczema, psoriasis, sensitive skin, and edema. Also, it has been reported that inflammatory conditions promote the growth of cancer cells.

Phytochemical compounds, present in any colorful fruit or vegetable, as well as a multitude of plants, have been reported to possess significant anti-oxidant and anti-inflammatory activity. These compounds include, but are not limited to, polyphenols and carotenoids. Polyphenols are plentiful in deep-colored plant foods such as blueberries, strawberries, grapes, green tea, and chocolate. Polyphenols that are particularly important as anti-oxidants and anti-inflammatories, are flavanoids, flavonoids and derivatives thereof, flavolignans, and curcumin. One class of carotenoids is responsible for the oranges and yellows in such foods as cantaloupe, mangoes, carrots, sweet potatoes and pink grapefruit. Another class of carotenoids, of which lycopene is one example, is responsible for the red in such foods as tomatoes and watermelon.

As noted, significant anti-oxidant and anti-inflammatory activity has been reported for those polyphenols that include flavanoids, flavonoids and derivatives thereof, flavolignans, and curcumin. Compounds of these groups are ubiquitous, being present in most plants. They have been studied since the 1940's, and their anti-oxidant activity is well documented at this point. It is believed that their anti-inflammatory activity stems from their anti-oxidant activity. A few of the many thousands of fruits, vegetables, and other plants and trees that are sources of these polyphenols are green tea, onions, apples, grapes, Ginkgo, and milk thistle (silybum marianum).

More specifically, polyphenols reported to be particularly useful, both as anti-oxidants and for treating inflammatory conditions, include the flavanoids: catechin and its derivatives; anthocyanin; oligomeric proanthocyanidins; the flavonoids and their derivatives: quercetin, kaempferol, myricetin, Ginkgo flavone glycosides, and quercetin chalcone; the flavolignans comprised in silymarin; and silybum marianum; and curcumin (see, e.g., Alt Med Rev, 1(2): 103-111, 1996). As one example, it is reported that sour cherries provide anti-inflammatory relief that is ten times stronger than that provided by aspirin, owing to the anthocyanin present therein.

Cacoa is one of the richest sources of flavanoids, including catechin and its derivative, epicatechin. Also, green tea extract is an excellent source of flavanoids derived from catechin and gallic acid, including catechin and its derivatives. The latter include epicatechin, gallocatechin and epigallocatechin, as well as their gallic acid esters such as epigallocatechin gallate (EGCG). These flavanoids make up about 30% by weight of dried green tea leaves. EGCG is the most prevalent, and has been shown to be the most effective agent against cutaneous inflammatory responses (see Archives of Dermatology, 136: 989-993, 990, August 2000). The anti-oxidant properties of these flavanoids are regarded as key to their skin protective qualities.

Preparations that take advantage of the antioxidant activity of flavanoids, flavonoids, and flavolignans to protect skin from photodamage, and that comprise catechins, silymarin and quercetin, are described in U.S. Pat. No. 5,804,168. It is also noted therein that oligomeric proanthocyanidins (found in grape seeds) have been used both topically and orally to protect the skin from various afflictions. Also, U.S. Pat. No. 5,616,332 suggests the use of green tea extract as an anti-inflammatory agent, in combination with sphinogosine, in topically applied, cosmetic skin-renewal stimulating compositions. It was reported therein that the agents are effective in controlling the long-term irritation typically produced by skin-renewal stimulating acids such as glycolic acid and other alpha and beta hydroxy acids commonly used.

It is believed that there is sufficient epidemiological, clinical and laboratory research to warrant the use of flavanoids to prevent and treat inflammatory conditions. (see, e.g., Alt Med Rev, 1(2): 103-111, 1996), and, based on extensive documented beneficial effects of green tea extracts on mouse skin models, many pharmaceutical and cosmetic companies are supplementing skin care products with green tea extracts. Also, initial studies using mouse models have indicated that topical application or oral consumption of green tea extract offers protection against inflammation (see Archives of Dermatology, 136: 989-993, 990, August 2000).

Pycnogenole® is a product marketed by Hankintatukku Natural Health Products Co., Helsinki, Finland, reported to be useful for treating and preventing inflammatory conditions by virtue of the flavanoids, contained therein, having the ability to inhibit the activity of certain enzymes that cause inflammation. More specifically, the flavanoids contained in Pycnogenole® are reported to bind to the skin proteins, collagen and elastin, and protect them from the enzymes of collagenase and elastase that are released during the inflammation process, and that would otherwise damage the above skin proteins.

In addition to having anti-inflammatory properties, flavanoids and flavonoids are reported to reduce hypersensitivity. In this regard, U.S. Pat. No. 5,616,332 notes that anti-oxidants tend to also function as anti-irritants. Also, U.S. Pat. No. 6,352,698 discloses the use of Ginkgo biloba, as well as green tea extracts, as active agents in compositions useful for treating hyper-reactive skin conditions, such as allergic type reactions or intolerance phenomena caused by external factors or factors intrinsic to the individual. In particular, U.S. Pat. No. 6,352,698 discloses that gingko extracts have excellent anti-inflammatory and anti-allergic activity which can be demonstrated on cutaneous cells such as keratinocytes and macrophages, and that compositions comprising green tea extract in combination with other agents produce an active hypoallergenic complex that lowers the reactivity threshold of the skin and decreases the magnitude of immunoallergic reactions.

The potential for flavanoids to have beneficial activity with respect to photoaging has also been reported. Photoaging is associated with oxidative stress and UV-induced skin injury. While the skin has an elaborate anti-oxidant defense system to combat UV-induced oxidative stress, excessive exposure to UV can overwhelm its oxidative capacity and cause oxidative damage and, ultimately, premature skin aging. Mouse studies have shown that oral ingestion or topical application of flavanoids from green tea can provide significant protection against UV-induced cutaneous edema and erythema. More specifically, topical application of EGCG has been shown to provide protection against UV-induced oxidative stress.

In addition, it is reported that clinical studies have shown that the catechin and the derivatives thereof, derived from green tea, have activity in preventing and treating hair loss by inhibiting the enzyme Type I 5-alpha reductase. The latter converts testosterone into DHT, a testosterone derivative associated with hair loss.

Yet another benefit of flavanoids, pertinent to skin, is reported in U.S. Pat. No. 6,375,992, namely, the hydration of mammalian skin and resultant alleviation of dry and flaky skin, as well as the improvement of its appearance by way of improving fine lines and wrinkles. Specifically, disclosed is the use of catechins and derivatives thereof, present in green tea and/or red grape extract, namely, catechin, gallocatechin, epicatechin, epigallocatechin, epigallocatechin gallate, and epicatechin gallate. Also reported therein is a synergistic enhancement of the mammalian skin hydrating effect of the catechin and catechin derivatives when combined with the juice or gel of a plant of the genus Aloe, or with glycerol.

As noted, a number of carotenoids also exhibit anti-oxidant and anti-inflammatory activity. There are hundreds of carotenoids. Those of particular interest as anti-oxidants and anti-inflammatory agents include lycopene, astaxanthin, lutein, alpha-carotene, beta-carotein, canthaxanthin, and zeaxanthin. Lycopene, the carotenoid that imparts the red color to tomatoes, watermelon, pink grapefruit, papaya, rosehips and guava, is an anti-oxidant capable of neutralizing oxygen free radicals that would otherwise damage cells, and inhibiting enzymes that generate such free radicals (85% of lycopene is found in tomatoes and tomato products). Lycopene has been described as the most powerful anti-oxidant of all the carotenoids. Studies have shown a relationship between a diet rich in tomato-based foods (rich in lycopene) and a reduced risk of cancer.

The ability of lycopene to inhibit cancer cell growth and shrink tumors has been attributed to its ability as an anti-oxidant to inhibit enzymes that cause inflammation, thereby, inhibiting inflammation processes that promote the growth of cancer cells. It has also been reported that, as an anti-oxidant, lycopene tends to have anti-inflammatory effect, because it neutralizes the pro-inflammatory effect of free radicals. Studies have shown that lycopene reduces oxidative stress that leads to heart disease and aging, in addition to cancer.

Astaxanthin is another carotenoid that is reported to be a potent anti-inflammatory and potentially useful for managing acute and chronic inflammation associated with certain diseases and conditions, including eczema and psoriasis. As an example, carrageenan-induced inflammation and subsequent edema in a rat paw caused by reactive oxygen molecules, was reported to be clearly and efficiently inhibited by astaxanthin, but not by Vitamin E (see Physiological Chemistry and Physics and Medical NMR, 22(1): 27-38, 1990). In one study, astaxanthin's anti-inflammatory activity was found to be more potent than that of the carotenoids: beta-carotene, canthazanthin, zeaxanthin, lutein and lycopene.

Anti-oxidant and anti-inflammatory activity has also been reported for lutein. Studies indicate that lutein may protect skin from oxidative stress induced by UV-A or UV-B radiation. For example, in one study, applying lutein to UV-B treated skin resulted in a 52% reduction of epidermal cell layers (hyper-proliferation of epidermal cells is characteristic of UV-B dermatitis). Also, in another study, the addition of lutein to skin after the application of a chemical irritant to the skin, resulted in a 50% reduction of erythema formation.

Generally, carotenoids are more readily absorbed by mammals in supplement form, than from foods. In foods, the carotenoids are, to a large extent, bound in a fibrous matrix that is difficult for the body to break down. Cooking helps. Also, lycopene present in heat-processed tomato juice is in the cis form and absorbed more readily by the body than lycopene present in unprocessed juice and in the trans form.

Peptide copper complexes having anti-oxidant and anti-inflammatory activity are disclosed in U.S. Pat. No. 5,118,665. Additionally, peptide copper complexes useful as anti-inflammatory agents and for wound healing, are disclosed in U.S. Pat. No. 4,760,051. Furthermore, the utility of such complexes as the active ingredient in cosmetic and skin treatment compositions is disclosed in U.S. Pat. Nos. 5,135,913 and 5,348,943. Peptide copper complexes that are useful for wound healing and skin health are also disclosed in U.S. Pat. Nos. 4,760,051; 4,665,054 and 4,877,770. Also, U.S. Pat. Nos. 5,177,061; 5,214,032; 5,120,831; 5,550,183 and 5,538,945 disclose peptide copper complexes that are beneficial for stimulating hair growth and preventing hair loss. The above U.S. patents, referenced in this paragraph are incorporated herein by reference in their entireties.

There remains a need in the art for compositions useful as anti-oxidants and anti-inflammatories that combine peptide copper complexes with phytochemical compounds, in particular, with flavanoids and carotenoids. Such compositions would, for example, mitigate tissue damage, disease and aging associated with oxidative stress; alleviate the discomfort, unsightliness, and promotion of disease, associated with inflammatory conditions. There also remains a need in the art for such compositions that are useful for the treatment and prevention of hair loss, and for improving the appearance of skin by improving fine lines and wrinkles, in addition to treating inflammation and hypersensitivity. The present invention fulfills these needs and provides further related advantages.

### BRIEF SUMMARY OF THE INVENTION

In brief, the present invention is directed to compositions having anti-oxidant, anti-inflammatory, and/or cosmetic utility when used for mammals, topically or internally, as well as to methods that use such compositions for enhancing the resistance of mammals to oxidative and inflammatory damage, for accelerating wound healing; for cosmetically treating mammalian skin to reduce the signs of aging and environmental exposure, and for stimulating hair growth, as well as preventing and treating hair loss.

In one embodiment, the present invention provides such compositions formed by combining at least one phytochemical compound with at least one peptide copper complex. Surprisingly, the anti-oxidant and anti-inflammatory activity of certain phytochemical compounds and copper peptide complexes is mutually and synergistically enhanced when the phytochemical compounds and copper peptide complexes are combined. In addition, the activity of phytochemical compounds and copper peptide complexes in healing wounds; in improving the appearance of skin by improving signs of aging and damage from oxidative stress (e.g., fine lines and wrinkles); and in stimulating hair growth and preventing or treating hair loss, is mutually and synergistically enhanced by way of the combination.

In other embodiments, directed to a composition of the present invention, the at least one phytochemical compound, combined with the at least one peptide copper complex, is a polyphenol, a carotenoid, and a mixture thereof, respectively. In other, more specific and related embodiments, respectively, the at least one polyphenol is a flavanoid, a flavonoid or derivative thereof, a flavolignan, and curcumin. Another embodiment is directed to compositions where the at least one phytochemical compound and the at least one peptide copper complex are encapsulated in liposomes or microsponges adapted to aid in delivery of the phytochemical compound and peptide copper complex, or to enhance the stability of the composition. In yet another embodiment, the components of the above-disclosed compositions are formulated in an instrument adapted to deliver the compounds via iontophoresis.

An additional embodiment of this invention is directed to the above compositions, further comprising a pharmaceutically-acceptable carrier and, thereby, adapted for oral or parenteral administration to a mammal. Yet other embodiments are adapted for topical application to mammalian skin, where the compositions further include, respectively, a pharmaceutically-acceptable diluent, a sunscreen agent, a skin conditioning agent, a skin protectant, an emollient, a humectant, an excipient, a textural modifier, an emulsifying agent, a preserving agent, a thickening agent, and a mixture thereof. These compositions may be in the form of a solution, cream, gel, fluid cream or milk, lotion, or oil.

The present invention is also directed to a method for enhancing or restoring the resistance of a mammal to oxidative or inflammatory damage, caused by the release of reactive oxygen species, and for accelerating wound healing, where the method comprises administering to the mammal, topically or internally, an effective amount of the disclosed inventive composition. In another embodiment, a method for cosmetically treating a mammal comprises administering an effective amount of the disclosed inventive composition, topically, orally, or parenterally. The effects of such cosmetic treatment include conditioning and smoothening the skin, as well as reducing the signs of photodamage and aging of the skin, and stimulating hair growth, as well as preventing or treating hair loss.

These and other aspects of this invention will be evident upon reference to the following detailed description of the invention.

### DETAILED DESCRIPTION OF THE INVENTION

As noted above, in one embodiment, disclosed is a composition having anti-oxidant, anti-inflammatory and/or cosmetic utility for a mammal, and formed by combining at least one phytochemical compound and at least one peptide copper complex. In specific embodiments, the phytochemical compound is a polyphenol, a carotenoid, and a mixture thereof, respectively. In more specific embodiments, the polyphenol is a flavanoid, a flavonoid or flavonoid derivative, a flavolignan, and curcumin, respectively. In yet more specific embodiments, respectively, the flavanoid is a catechin or catechin derivative, oligomeric proanthocyanidin, and anthocyanin; the flavonoid or flavonoid derivative is quercetin, kaempferol, myricetin, baicalein, rutin, Ginkgo flavone glycoside, and quercetin chalcone; and the flavolignan is silybin, silydianin, and silychristin. silybum marianum, rutin, baicalein, and curcumin.

As used herein, the expression “phytochemical compound” refers to any of the anti-oxidant pigments that are naturally present in, and impart color to fruits and vegetables, as well present in the roots, bark, leaves, flowers and seeds of plants. Polyphenols and carotenoids are examples of phytochemical compounds. Also, as used herein, the term “polyphenol” refers to a water soluble phytochemical compound characterized by having a molecular weight of less than about 3000 and by having more than one phenolic group. Polyphenols include flavanoids and derivatives thereof; flavonoids and derivatives thereof; flavolignans gallic acid and its esters; phlorotannins; and the esters, glycosides and amides of the hydroxycinnamic acids.

Flavanoids, flavonoids and their derivatives, flavolignans, and polyphenolic rhizomes represent some of the more significant polyphenols, with regard to having potent anti-oxidant and anti-inflammatory properties. The principal types of flavanoids, and those that have been reported to be the most potent anti-oxidants and anti-inflammatories of the flavanoids, are based on the flavan-3-ol nucleus, shown below as formula I.

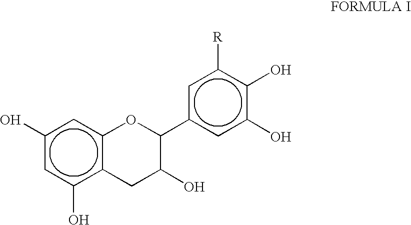

When R is hydrogen, then the flavanoid is a catechin or epicatechin. When is R is an OH group, then the flavanoid is a gallocatechin or epigallocatechin. Whether the flavanoid is a catechin or an epicatechin depends on the conformation of the OH group in the center, oxygen-bearing ring. Accordingly, catechin, gallocatechin, epicatechin, and epigallocatechin are shown below as Formulas II, III, IV and V, respectively.

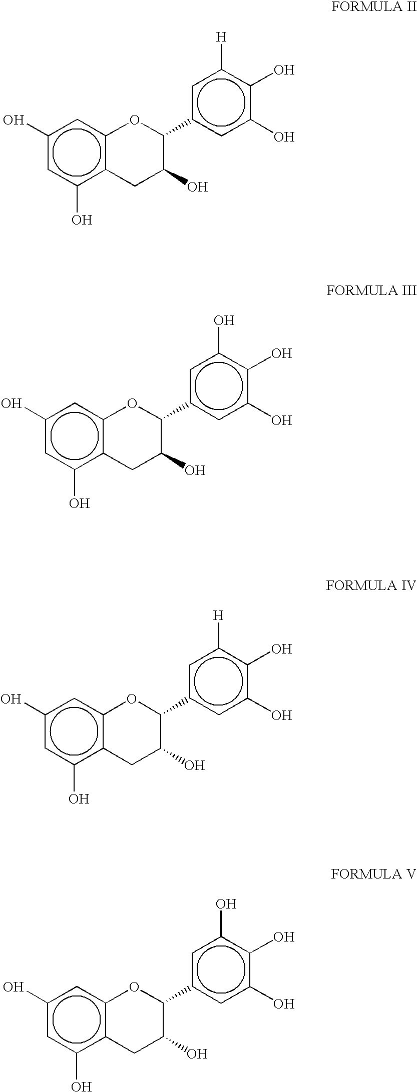

As noted previously, a major source of the above-described catechin and catechin derivatives is cacoa and tea solids, in particular, green tea solids. As used herein, “tea solids” refers to solids obtained from, for example, the genus Camellia, including Camellia sinensis (i.e., green tea) and Camellia assaimica, where the solids include freshly gathered tea leaves, fresh tea leaves which are dried immediately after gathering, fresh tea leaves that have been heat-treated before drying to inactivate any enzymes present, unfermented tea, fermented tea, instant green fermented tea, partially fermented tea leaves, and aqueous extracts of these leaves.

A catechin derivative that is reported to be the most prevalent in green tea, and the most effective anti-oxidant, is epigallocatechin gallate, an ester of epigallocatechin and gallic acid. This catechin derivative is shown below as Formula VI.

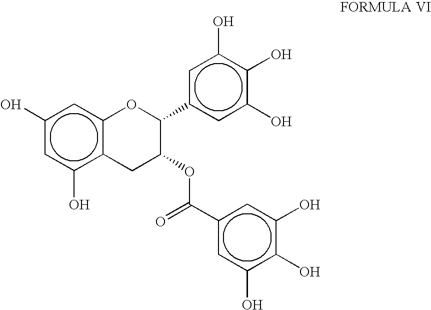

Catechin or catechin derivative monomers can link to form polymers. Oligomers of up to about six units are generally soluble in water. Oligomeric proanthocyanidins are oligomeric flavanoids that are usually dimers and trimers of monomers based on the flavan 3-ol nucleus. They are present, for example, in the bark of pine trees, grape seeds and skins, cranberries, tea solids, and cacao. A dimer of epicatechin that is found in cacoa is shown below as Formula VII.

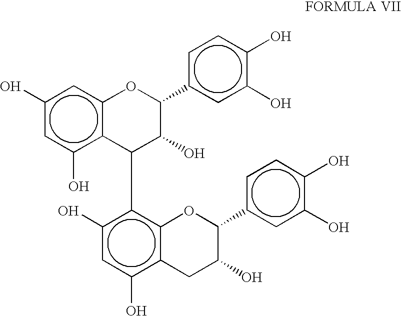

The above catechin and catechin derivatives are commercially available. For example, many of these compounds are available from Sigma-Aldrich Co., St. Louis, Mo. Also, they can be provided in extracts of the above teas, or extracted therefrom. Suitable methods for obtaining tea extracts and extracting polyphenols, including flavenoids, from tea solids are well known to those skilled in the art and described, for example, in U.S. Pat. Nos. 6,375,992; 4,935,256; 4,680,193; and 4,668,525 incorporated herein by reference in their entireties.

In one particular embodiment of the present invention, directed to compositions comprising at least one copper peptide complex in combination with at least one phytochemical compound that is a flavanoid, the flavanoid is anthocyanin. The latter is a soluble glucoside natural dye (plant pigment), found in the fruits, leaves and blossoms of higher plants. For example, anthocyanin imparts the blue and purple color to blueberries, plums and cherries. It is shown below as Formula VIII.

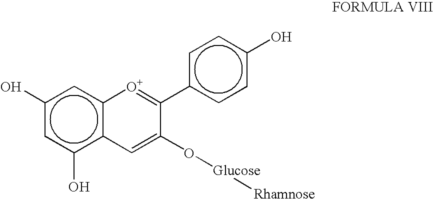

The present invention is also directed to compositions wherein at least one copper peptide complex is combined with at least one flavonoid or derivative thereof. Certain of the flavonoids can be shown by the general formula shown below as Formula IX.

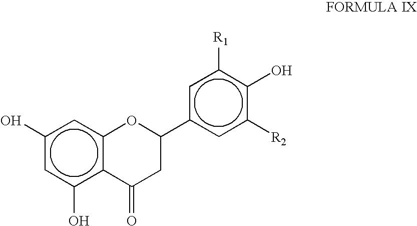

When R₁ and R₂ are hydrogen, the flavonoid is kaemferol. When R₁ is an OH group, and R₂ is hydrogen, the flavonoid is quercetin. When each of R₁ and R₂ is an OH group, the flavonoid is myricetin. Another of the flavonoids is rutin (extracted from the fruit of the Fava D'Anta tree, native to the savanna areas of the northern and eastern regions of Brazil) which is an ester of quercetin. Yet another of the flavonoids is baicalein, shown below as Formula X.

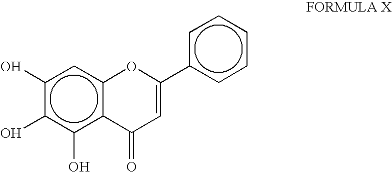

One of the above-mentioned flavonoid derivatives is quercetin chalcone, shown below as Formula XI.

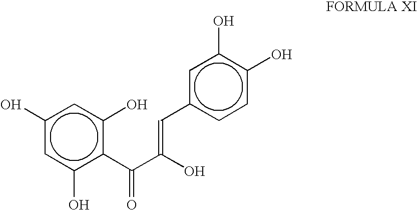

As shown, quercetin chalcone is structurally similar to the above flavonoids, differing in that the center ring has been opened and the oxygen therein converted to an OH group. In fact, it is derived from quercetin, the latter being chemically modified so as to be more readily absorbed by the body. Another of the flavonoid derivatives is Ginkgo flavone glycoside, extracted from the Ginkgo biloba tree.

Another particular embodiment of the present invention is directed to a composition wherein at least one peptide copper complex is combined with a flavolignan. In a more specific related embodiment, the flavolignan is silymarin. Silymarin is actually a mixture of the flavolignans: silybin (the most prevalent component), silydianin, and silychristin. These anti-oxidants, extracted from milk thistle (silybum marianum), have been shown to inhibit lipid peroxidation (see, e.g., Journal of Applied Toxicology, 12: 439-442, 1992).

In yet another particular embodiment of the present invention, the at least one peptide copper complex is combined with a polyphenolic rhizome. In a more specific, related embodiment, the polyphenolic rhizome is curcumin, a rhizome of the ginger family. Curcumin is shown below as Formula XII.

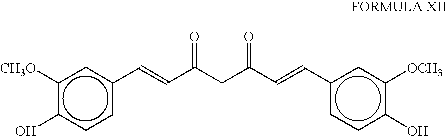

The above flavanoids, flavonoids and derivatives thereof, flavolignans, and polyphenolic rhizomes are commercially available and/or provided in extracts of, or extracted from, fruits, vegetables, and other plants, using methods well known to those skilled in the art and similar to the methods described above and used to provide catechin and catechin derivatives.

Another embodiment of the present invention, directed to anti-oxidative and anti-inflammatory compositions, combines at least one copper peptide complex with at least one carotenoid. In related, more specific embodiments, the carotenoid is lycopene, astaxanthin, lutein, alpha-carotene, beta-carotene, canthaxanthin, and zeaxanthin. To those skilled in the art, carotenoids constitute a well-known and well-defined, large class of pigments occurring in the tissues of higher plants, algae, and bacteria, as well as fungi. A number of carotenoids, including those listed above, are commercially available and/or extracted from their natural sources, or otherwise produced, by methods well known in the art. For example, alpha and beta-carotene are extracted from carrots and palm oil, or concentrated by a chromatographic process from alfalfa. Beta-carotene can also be made by a microbial fermentation process from corn and soybean oil. Lutein is present in, and can be extracted from peas, carrots, and squashes.

As another example, lycopene can be extracted from tomatoes. The lycopene molecule, which has the molecular formula C₄₀H₅₆, is shown below as Formula XIII.

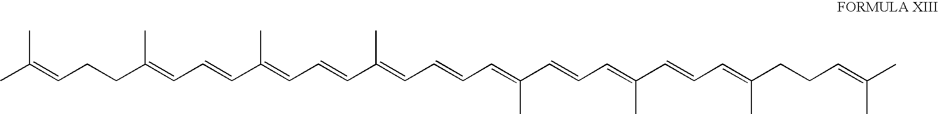

As noted, the compositions of the present invention include at least one peptide copper complex. As used herein, the term “peptide copper complex” refers to a coordination compound comprising a peptide molecule and a copper ion non-covalently complexed therewith. The peptide molecule serves as the complexing agent by donating electrons to the copper ion to yield the non-covalent complex. The peptide molecule is a chain of two or more amino acid units covalently bonded together via amide linkages (for example, —CONH—), the formation of such linkages being accompanied by the elimination of water. The amino acid units are from amino acids that are naturally occurring or otherwise. Also, at least one amide linkage nitrogen atom may have covalently bonded thereto either a hydrogen atom or another moiety.

Generally, an amino acid consists of an amino group, a carboxyl group, a hydrogen atom, and an amino acid side-chain moiety—all bonded, in the case of an alpha-amino acid, to a single carbon atom that is referred to as an alpha-carbon (see example shown below). The amino acid units of the peptide copper complexes comprised in compositions of the present invention may be provided by amino acids other alpha-amino acids. For example, the amino acids may be beta- or gamma-amino acids, such as those shown below.

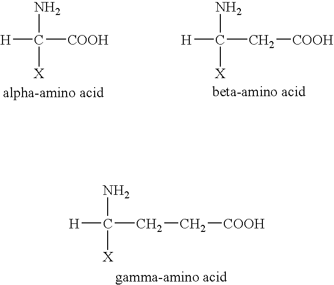

where X is the amino acid side-chain moiety.

Naturally occurring amino acids, that is, amino acids from which the amino acid units of naturally occurring proteins are derived, and their respective naturally occurring, amino acid side chain moieties, are shown below in Table 1. In addition to the naturally occurring L amino acids, D amino acids may also be used as constituents of the peptide.

TABLE 1
NATURALLY OCCURRING AMINO ACID SIDE-CHAIN MOIETIES

| Amino Acid Side Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| 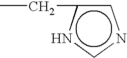 | Histidine |
| —CH₂COO— | Aspartic Acid |
| —CH₂CH₂COO— | Glutamic Acid |
| —CH₂CONH₂ | Asparagine |
| —CH₂CH₂CONH₂ | Glutamine |
| 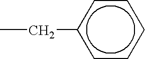 | Phenylalanine |
| 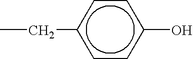 | Tyrosine |
| 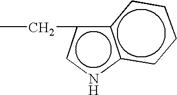 | Tryptophan |
| —CH₂SH | Cysteine |
| —CH₂CH₂SCH₃ | Methionine |
| —CH₂OH | Serine |
| —CH(OH)CH₃ | Threonine |

One example of a copper peptide complex is alanyl-histidyl-lysine:copper(II). Copper(II), as is well understood by the skilled artisan, designates a copper ion having a valence of 2 (e.g., Cu⁺²). Additional examples of the peptide copper complexes, encompassed in embodiments of the present invention, include, but are not limited to, those disclosed and described in the above-cited U.S. patents, directed to peptide copper complexes, that have been incorporated herein by reference.

Further, the expression “peptide copper complex,” as used herein, encompasses peptide copper complex derivatives. The expression “peptide copper complex derivative,” as used herein, refers to a peptide copper complex where the peptide molecule thereof has: 1) at least one amino acid side chain moiety that is a modification and/or variation of a naturally occurring, amino acid side-chain moiety; and/or 2) at least one of the hydrogens, bonded to an amide linkage nitrogen atom, substituted with a different moiety; and/or 3) the carboxyl group of the carboxyl terminal residue esterified or otherwise modified; and/or 4) at least one hydrogen, bonded to the nitrogen atom of the amino-terminal residue, substituted with a different moiety.

For example, the amino acid side-chain moieties of alanine, valine, leucine, isoleucine and phenylalanine may generally be classified as lower chain alkyl (1-12 carbon atoms), lower chain aryl (6-12 carbon atoms), or lower chain aralkyl (7-12 carbon atoms) moieties. The amino acid side-chain moieties of the peptide copper complex derivatives, may include other straight chain or branched, cyclic or noncyclic, substituted or unsubstituted, saturated or unsaturated lower chain alkyl, aryl or aralkyl moieties. Also, the peptide copper complex derivative may, for example, be N-alkylated at one or more peptide bonds; and/or its carboxyl terminus may be esterified, for example, with a methyl, ethyl, or benzyl group, or may be reduced to a hydroxy or aldehyde. Additionally, the peptide copper complex derivative may, for example, be N-alkylated, N-acylated or N-sulfonylated at the amino terminus with, for example, methyl, benzyl, acetyl, benzoyl, methanesulfonyl, or fluorenyloxycarbonyl moieties.

Examples of the peptide copper complex derivatives, encompassed in embodiments of the present invention, include, but are not limited to, those disclosed and described in the above-cited U.S. Patents, directed to peptide copper complexes, that have been incorporated herein by reference; as well as those disclosed and described in the published PCT application having the international publication number WO 94/03482, incorporated herein by reference in its entirety.

In certain specific embodiments of the composition of the present invention, the at least one peptide copper complex is alanyl-histidyl-lysine:copper(II) (“AHK—Cu”), valyl-histidyl-lysine:copper(II) (“VHK—Cu”), or glycyl-histidyl-lysine:copper(II) (GHK—Cu”), respectively. As is well understood in the art, copper(II) designates a copper ion having a valence of 2 (e.g., Cu⁺²). Further, such peptides may be in either the L or D form. In a related, more specific embodiment, they are all in the L form.

In another specific embodiment, the composition of the present invention includes the peptide copper complex derivative that is a derivative of GHK—Cu having the general formula:

[glycyl-histidyl-lysine-R]:copper(II)

where R is an alkyl moiety containing from one to eighteen carbon atoms, an aryl moiety containing from six to twelve carbon atoms, an alkoxy moiety containing from one to twelve carbon atoms, or an aryloxy moiety containing from six to twelve carbon atoms. This derivative of GHK—Cu is further described in the above-cited U.S. patents that are incorporated herein by reference in their entireties.

The disclosed compositions may be prepared from aqueous solutions of peptide copper complexes. Such solutions are prepared by methods that are well known to those skilled in the art. For example, an amount of dried peptide copper complex suitable for a desired concentration is readily dissolved in water with mixing and gentle heating. An alternative method is to prepare a solution of the desired peptide, followed by the addition of a copper salt in the desired molar ratio to yield the desired solution of the peptide copper complex. Examples of copper salts that may be used are cupric chloride and cupric acetate. When aqueous solutions of peptide copper complexes are prepared, the solutions are neutralized, typically with NaOH.

In various embodiments of the inventive composition of the present invention, the concentration of the at least one peptide copper complex, by weight of the composition, ranges from about 0.01% to about 5%, from about 0.025% to about 1%, and from about 0.05% to about 0.5%, respectively. Also, the molar ratio of peptide to copper in the complex ranges from about 1:1 to about 3:1 in some embodiments, and from about 1:1 to about 2:1 in other embodiments.

One embodiment is directed to a composition of the present invention that further includes a pharmaceutically-acceptable encapsulating coating or carrier, thus being suitable for oral or parenteral administration to the body. The coating or carrier should not interact with peptide copper complex or phytochemical compound so as to significantly reduce the effectiveness thereof. An effective dosage of this embodiment delivers approximately 0.01 to 10 mg of peptide copper complex per kg of body weight. Methods for encapsulating compositions (such as in a coating of hard gelatin) for oral administration are well known in the art (Baker, Richard, Controlled Release of Biological Active Agents. John Wiley and Sons, 1986, incorporated herein by reference in its entirety). Suitable pharmaceutically-acceptable carriers for parenteral application, such as intravenous, subcutaneous or intramuscular injection, include sterile water, physiological saline, bacteriostatic saline (saline containing 0.9 mg/ml benzyl alcohol) and phosphate-buffered saline.

The present invention, in another embodiment, is also directed to a composition, topically applied to mammalian skin and formed by combining at least one peptide copper complex with at least one phytochemical compound, where the combined compounds are encapsulated in liposomes or microsponges to aid in the delivery of the peptide copper complex or to increase the stability of the composition. In yet another embodiment of such compositions, the combined compounds may be formulated in an instrument allowing the delivery of the compounds via iontophoresis.

Another embodiment, directed to a composition of the present invention adapted for topical application to mammalian skin, further comprises a pharmaceutically-acceptable diluent, which, in exemplary related embodiments that are liquids, is saline and sterile water, respectively. Exemplary embodiments that are lotions, creams, and gels, respectively, include additional ingredients to impart the desired texture, consistency, viscosity, and appearance. Such ingredients are familiar to those skilled in the art and include, for example, a petrolatum based cream, a pharmaceutically-acceptable gel, a short chain alcohol, or a short chain glycol. Some examples of compositions useful for cleansing, protecting and treating skin are: creams for the face, hands, feet, or the entire body (i.e., day creams, night creams, make-up removal creams, and foundation creams); make-up removal formulations; protective or skin care body milks; skin care lotions, gels, or foams (such as cleansing or disinfecting lotions); bath compositions; deodorant compositions; and aftershave and preshave gels or lotions.

The compositions of the present invention, adapted for topical application to the skin, may also contain at least one active ingredient, in addition to the at least one phytochemical compound and the at least one peptide copper complex. Active ingredients, as defined herein, are compounds that provide benefits to the skin and/or desirable properties to cosmetic formulations. Some examples of active ingredients are sunscreens and tanning agents, skin conditioning agents, skin protectants, emollients and humectants.

The sunscreen agents that are included in certain embodiments of the compositions of the present invention are active ingredients that absorb, reflect, or scatter radiation in the UV range at wavelengths from 290 to 400 nanometers. Specific examples include benzophenone-3 (oxybenzone), benzophenone-4 (sulisobenzone), benzophenone-8 (dioxybenzone), butyl methoxydibenzoylmethane (Avobenzone), DEA-methoxycinnamate (diethanolamine methoxycinnamate), ethyl dihydroxypropyl PABA (ethyl 4-[bis(hydroxypropyl)] aminobenzoate), ethylhexyl dimethyl PABA (Padimate O), ethylhexyl methoxycinnamate (octyl methoxycinnamate), ethylhexyl salicylate (octyl salicylate), homosalate, menthyl anthranilate (Meradimate), octocrylene, PABA (aminobenzoic acid), phenylbenzimidazole sulfonic acid (Ensulizole), TEA-salicylate (trolamine salicylate), titanium dioxide, and zinc oxide. One skilled in the art will appreciate that other sunscreen agents may be used in the compositions and preparations of the present invention.

Other embodiments of the topically applied compositions disclosed herein, contain skin conditioning agents. The latter agents include substances that enhance the appearance of dry or damaged skin, as well as materials that adhere to the skin to reduce flaking, restore suppleness, and generally improve the appearance of skin. Representative examples of the skin conditioning agents that may be used include: acetyl cysteine, N-acetyl dihydrosphingosine, acrylates/behenyl acrylate/dimethicone acrylate copolymer, adenosine, adenosine cyclic phosphate, adensosine phosphate, adenosine triphosphate, alanine, albumen, algae extract, allantoin and derivatives, aloe barbadensis extracts, aluminum PCA, amyloglucosidase, arbutin, arginine, azulene, bromelain, buttermilk powder, butylene glycol, caffeine, calcium gluconate, capsaicin, carbocysteine, carnosine, beta-carotene, casein, catalase, cephalins, ceramides, chamomilla recutita (matricaria) flower extract, cholecalciferol, cholesteryl esters, coco-betaine, coenzyme A, corn starch modified, crystallins, cycloethoxymethicone, cysteine DNA, cytochrome C, darutoside, dextran sulfate, dimethicone copolyols, dimethylsilanol hyaluronate, DNA, elastin, elastin amino acids, epidermal growth factor, ergocalciferol, ergosterol, ethylhexyl PCA, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, and kinetin.

Other examples of skin conditioning agents that may be used for the topically applied compositions of this invention are: lactoferrin, lanosterol, lauryl PCA, lecithin, linoleic acid, linolenic acid, lipase, lysine, lysozyme, malt extract, maltodextrin, melanin, methionine, mineral salts, niacin, niacinamide, oat amino acids, oryzanol, palmitoyl hydrolyzed proteins, pancreatin, papain, PEG, pepsin, phospholipids, phytosterols, placental enzymes, placental lipids, pyridoxal 5-phosphate, quercetin, resorcinol acetate, riboflavin, RNA, saccharomyces lysate extract, silk amino acids, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, and zinc gluconate. The compositions of the present invention, suitable for topical application to skin, may contain skin conditioning agents other than those listed above, as may be readily appreciated by one skilled in the art.

Other related embodiments include at least one skin protectant (defined, as the term is used herein, as a compound that protects injured or exposed skin or mucous membrane surfaces from harmful or irritating external compounds), other than that provided by the phytochemical compound or peptide copper complex. Representative examples include: algae extract, allantoin, aluminum hydroxide, aluminum sulfate, betaine, cerebrosides, dimethicone, glucuronolactone, glycerin, kaolin, lanolin, malt extract, mineral oil, petrolatum, potassium gluconate, and talc. One skilled in the art will readily appreciate that skin protectants other than those listed above may also be used in related embodiments of the present invention.

Yet further related embodiments contain one or more emollients. An emollient, as the term is used herein, is a cosmetic ingredient that can help skin maintain a soft, smooth, and pliable appearance. Emollients are able to provide these benefits, largely owing to their ability to remain on the skin surface or in the stratum corneum to act as a lubricant and reduce flaking. Some examples of emollients, suitable for embodiments of this invention, are: acetyl arginine, acetylated lanolin, algae extract, apricot kernel oil PEG-6 esters, avocado oil PEG-11 esters, bis-PEG-4 dimethicone, butoxyethyl stearate, C₁₈-C₃₆ acid glycol ester, C₁₂-C₁₃ alkyl lactate, caprylyl glycol, cetyl esters, cetyl laurate, coconut oil PEG-10 esters, di-C₁₂-C₁₃ alkyl tartrate, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, hydroxypropyl bisisostearamide MEA, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyldodecanol, odontella aurita oil, 2-oleamido-1,3 octadecanediol, palm glycerides, PEG avocado glycerides, PEG castor oil, PEG-22/dodecyl glycol copolymer, PEG shorea butter glycerides, phytol, raffinose, stearyl citrate, sunflower seed oil glycerides, and tocopheryl glucoside. One skilled in the art will readily appreciate that other emollients may also be used in the embodiments of the present invention that are suitable for topical application to mammalian skin.

Humectants may also be included in the compositions of related embodiments of this invention. Humectants are cosmetic ingredients that help maintain moisture levels in skin. Some examples of suitable humectants are: acetyl arginine, algae extract, aloe barbadensis leaf extract, betaine, 2,3-butanediol, chitosan lauroyl glycinate, diglycereth-7 malate, diglycerin, diglycol guanidine succinate, erythritol, fructose, glucose, glycerin, honey, hydrolyzed wheat protein/PEG-20 acetate copolymer, hydroxypropyltrimonium hyaluronate, inositol, lactitol, maltitol, maltose, mannitol, mannose, methoxy PEG, myristamidobutyl guanidine acetate, polyglyceryl sorbitol, potassium PCA, propylene glycol, sodium PCA, sorbitol, sucrose, and urea. Other humectants may be used for embodiments of this invention, as will be appreciated by one skilled in the art.

In yet further related embodiments, compositions of the present invention may contain additional ingredients such as fatty alcohols, fatty acids, organic or inorganic bases, preserving agents, wax esters, steroid alcohols, triglyceride esters, phospholipids such as lecithin and cephalin, polyhydric alcohol esters, fatty alcohol ethers, hydrophilic lanolin derivatives, hydrophilic beeswax derivatives, cocoa butter waxes, silicon oils, pH balancers, cellulose derivatives, and hydrocarbon oils such as palm oil, coconut oil, and mineral oil. Additional ingredients that are particularly useful, as is well understood by those skilled in the art, are those that may be used to vary the texture, viscosity, color and appearance of the above compositions and preparations, and include emulsifying agents, thickening agents, and surfactants.

Emulsifiers and surfactants are used in preparing those embodiments of the present invention directed to compositions for topical application to skin that are formulated as emulsions. Either water in oil or oil in water emulsions may be formulated. Examples of suitable surfactants and emulsifying agents include: nonionic ethoxylated and nonethoxylated surfactants, abietic acid, almond oil PEG, beeswax, butylglucoside caprate, C₁₈-C₃₆ acid glycol ester, C₉-C₁₅ alkyl phosphate, caprylic/capric triglyceride PEG-4 esters, ceteareth-7, cetyl alcohol, cetyl phosphate, corn oil PEG esters, DEA-cetyl phosphate, dextrin laurate, dilaureth-7 citrate, dimyristyl phosphate, glycereth-17 cocoate, glyceryl erucate, glyceryl laurate, hydrogenated castor oil PEG esters, isosteareth-11 carboxylic acid, lecithin, lysolecithin, nonoxynol-9, octyldodeceth-20, palm glyceride, PEG diisostearate, PEG stearamine, poloxamines, polyglyceryls, potassium linoleate, PPG's, raffinose myristate, sodium caproyl lactylate, sodium caprylate, sodium cocoate, sodium isostearate, sodium tocopheryl phosphate, steareths, TEA-C₁₂-C₁₃ pareth-3 sulfate, tri-C₁₂-C₁₅ pareth-6 phosphate, and trideceths. Other surfactants and emulsifiers may be used, as will be appreciated by one skilled in the art.

Additional related embodiments further include thickening or viscosity increasing agents. Suitable examples include those agents commonly used in skin care preparations, such as: acrylamides copolymer, agarose, amylopectin, bentonite, calcium alginate, calcium carboxymethyl cellulose, carbomer, carboxymethyl chitin, cellulose gum, dextrin, gelatin, hydrogenated tallow, hydroxytheylcellulose, hydroxypropylcellulose, hydroxpropyl starch, magnesium alginate, methylcellulose, microcrystalline cellulose, pectin, various PEG's, polyacrylic acid, polymethacrylic acid, polyvinyl alcohol, various PPG's, sodium acrylates copolymer, sodium carrageenan, xanthan gum, and yeast beta-glucan. Thickening agents other than those listed above may also be used in embodiments of this invention.

As noted above, those embodiments of the present invention that are intended primarily as products for topical application to mammalian skin are typically in the form of a cream, gel, fluid cream or milk, lotion, or oil. Also, the compositions may be further combined with suitable excipients adapted for application to the face and neck. Suitable excipients should have a high affinity for the skin, be well tolerated, stable, and yield a consistency that allows for easy and pleasant utilization.

A further aspect of the present invention is a method for restoring the resistance of a mammal to oxidative or inflammatory damage caused by the release of reactive oxygen species, the method comprising orally, parenterally, or topically administering to the mammal a therapeutically effective amount of a composition of the present invention. The expression “reactive oxygen species” refers to the superoxide anion (O₂), hydrogen peroxide, hydroxyl radical, and lipid peroxides—among other species.

Another aspect of the present invention is a method for accelerating the healing of wounds in a mammal, the method comprising orally, parenterally, or topically administering to the mammal a therapeutically effective amount of a composition of the present invention.

This invention is further directed to a method for treating mammalian skin to condition and smoothen the skin, lessen hyperpigmentation, and prevent or reduce the appearance of fine lines and wrinkles, and other signs of photodamage and aging of the skin. The method comprises contacting the skin with an effective amount of an above-disclosed composition. As a specific example, a small amount of material (from about 1 to about 5 ml) is applied to exposed areas of skin from a suitable container or applicator, and, if necessary, the material is then spread over and/or rubbed into the skin using the hand or finger, or a suitable device. Each of the compositions and preparations disclosed herein is typically packaged in a container to suit its viscosity and intended use by the consumer. For example, a lotion or fluid cream may be packaged in a bottle, roll-ball applicator, capsule, propellant-driven aerosol device, or a container fitted with a manually operated pump. A cream can simply be stored in a non-deformable bottle or squeeze container, such as a tube or a lidded jar.

Finally, yet another aspect of the present invention is a method for stimulating hair growth, preventing hair loss, or treating hair loss, the method comprising orally, parenterally, or topically administering to a mammal a therapeutically effective amount of a composition of the present invention.

The following examples are provided for the purpose of illustration, not limitation.

### EXAMPLES

The examples which follow illustrate the preparation, characterization and utility of certain exemplary embodiments of the present invention.

### Example 1

The resulting moisturizing lotion incorporating one embodiment of the composition of this invention would contain the following ingredients.

| | Preferred | |
|---|---|---|
| Ingredients | weight % | Range |
| Water | 74.0% | 50% to 80% |
| Glycerin | 1.00% | 0.01% to 25% |
| xanthan gum | 0.50% | 0.01% to 25% |
| diisopropyl adipate | 4.00% | 0.01% to 25% |
| isocetyl stearate | 6.00% | 0.01% to 25% |
| octyl palmitate | 10.00% | 0.01% to 25% |
| glyceryl stearate | 1.00% | 0.01% to 10% |
| cetyl alcohol | 1.00% | 0.01% to 10% |
| stearyl alcohol | 0.80% | 0.01% to 10% |
| behenyl alcohol | 0.50% | 0.01% to 10% |
| palmitic acid | 0.30% | 0.01% to 10% |
| stearic acid | 0.25% | 0.01% to 10% |
| glycyl-L-histidyl-L-lysine copper complex | 0.20% | 0.01% to 10% |
| Catechin | 0.01% | 0.001% to 10% |
| Gallocatechin | 0.01% | 0.001% to 10% |
| Epicatechin | 0.01% | 0.001% to 10% |
| propylene glycol | 0.55% | 0.001% to 10% |
| diazolidinyl urea | 0.03% | 0.001% to 10% |
| iodopropynyl butylcarbonate | 0.02% | 0.001% to 10% |

This formulation is beneficial as the phytochemical compound provides anti-inflammatory action to the skin in addition to the anti-inflammatory and tissue rebuilding activity provided by the presence of the copper peptide compound. This type of formulation would sooth, protect, and restore the youthful appearance of the skin.

### Example 2

The resulting moisturizing cream incorporating one embodiment of the composition of this invention would contain the following ingredients.

| | Preferred | |
|---|---|---|
| Ingredients | weight % | Range |
| purified water | 76.35% | 50% to 80% |
| ethylhexyl palmitate | 8.00% | 0.01% to 25% |
| octyldodecanol | 2.50% | 0.01% to 25% |
| butyloctyl calicylate | 2.00% | 0.01% to 25% |
| squalane | 1.50% | 0.01% to 25% |
| jojoba oil | 0.50% | 0.01% to 10% |
| tocopheryl acetate | 0.20% | 0.01% to 10% |
| bisabolol | 0.20% | 0.01% to 10% |
| polyacrylamide | 1.50% | 0.01% to 10% |
| laureth-7 | 0.50% | 0.01% to 10% |
| glycerin | 3.00% | 0.01% to 25% |
| panthenol | 0.60% | 0.01% to 10% |
| allantion | 0.10% | 0.01% to 10% |
| cyclomethicone | 0.50% | 0.01% to 10% |
| carbomer | 0.10% | 0.01% to 10% |
| polysorbate 20 | 0.20% | 0.01% to 10% |
| glycyl-L-histidyl-L-lysine copper complex | 0.25% | 0.01% to 5% |
| Lycopene | 1.00% | 0.001% to 10% |
| propylene glycol | 0.56% | 0.001% to 10% |
| diazolidinyl urea | 0.30% | 0.001% to 10% |
| methylparaben | 0.11% | 0.001% to 10% |
| propylparaben | 0.03% | 0.001% to 10% |

This formulation is beneficial as the phytochemical compound provides anti-inflammatory and protective action to the skin in addition to the anti-inflammatory and tissue rebuilding activity provided by the presence of the copper peptide compound. This type of formulation would sooth, protect, and restore the youthful appearance of the skin.

### Example 3

The resulting body lotion incorporating one embodiment of the composition of this invention would contain the following ingredients.

| | Preferred | |
|---|---|---|
| Ingredients | weight % | Range |
| water | 74.35% | 50% to 80% |
| hydrogenated vegetable oil | 6.00% | 0.01% to 25% |
| canola oil | 4.00% | 0.01% to 25% |
| polyoxyethylene stearyl stearate | 4.00% | 0.01% to 25% |
| steareth-21 | 2.00% | 0.01% to 25% |
| octyldodecanol | 6.00% | 0.01% to 25% |
| sorbeth-30 | 2.50% | 0.01% to 25% |
| glycyl-L-histidyl-L-lysine copper complex | 0.10% | 0.01% to 10% |
| Catechin | 0.02% | 0.001% to 10% |
| Gallocatechin | 0.02% | 0.001% to 10% |
| Epicatechin | 0.01% | 0.001% to 10% |
| propylene glycol | 0.56% | 0.001% to 10% |
| diazolidinyl urea | 0.30% | 0.001% to 10% |
| methylparaben | 0.11% | 0.001% to 10% |
| propylparaben | 0.03% | 0.001% to 10% |
| Total | 100.00% | |

This formulation is beneficial as the phytochemical compounds provide anti-inflammatory and protective action to the skin in addition to the anti-inflammatory and tissue rebuilding activity provided by the presence of the copper peptide compound. This type of formulation would sooth, protect, and restore the youthful appearance of the skin.

### Example 4

The resulting hair treatment composition incorporating one embodiment of the composition of this invention would contain the following ingredients.

| | Preferred | |
|---|---|---|
| Ingredients | weight % | Range |
| Water | 97.94% | 50% to 80% |
| Sodium Chloride | 0.9% | 0.01% to 25% |
| L-alanyl-L-histidyl-L-lysine copper complex | 0.20% | 0.01% to 10% |
| Catechin | 0.02% | 0.001% to 10% |
| Gallocatechin | 0.02% | 0.001% to 10% |
| Epicatechin | 0.01% | 0.001% to 10% |
| propylene glycol | 0.56% | 0.001% to 10% |
| Phenoxyethanol | 0.30% | 0.001% to 10% |
| Isopropylparaben | 0.02% | 0.001% to 10% |
| Isobutylparaben | 0.03% | 0.001% to 10% |
| Total | 100.00% | |

This formulation is beneficial as the phytochemical compounds provide anti-inflammatory and protective action to the skin in addition to the anti-inflammatory and tissue rebuilding and follicle-stimulating activity provided by the presence of the copper peptide compound. This type of formulation would sooth, protect, and restore the youthful appearance of the skin.

### Example 5

The efficacy of the disclosed compositions of this invention can be demonstrated via standard assays used to assess the performance of such compositions. For example, the compositions of this invention can be provided to volunteer subjects having signs of photo damaged skin such as age spots, hyperpigmentation, fine lines and wrinkles. These signs of clinical aging could be rated using, for example, a scale of 0-9 at baseline, and at weeks 4 and 8. Subjects could be given compositions suitable for topical application, formulated according to the present invention, along with instructions that the compositions are to be topically applied twice daily to the areas showing signs of photodamage and aging. Clinical photographs may also be taken for comparison.

At the end of 4 and 8 weeks, the clinical signs of aging would again be assessed, and corresponding photographs taken for comparison with those taken earlier and subsequently. Comparison of data with the data collected earlier and subsequently would reveal a diminishment of the clinical signs of aging and photodamaged skin as a result of the treatment with the composition with the skin care compositions and preparations of this invention.

All of the U.S. patents, U.S. patent application publications, U.S. patent applications, foreign patents, foreign patent applications and non-patent publications listed in the Application Data Sheet, are incorporated herein by reference in their entirety.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A composition comprising a peptide copper complex and a phytochemical compound.

2. The composition of claim 1 wherein the phytochemical compound is a polyphenol.

3. The composition of claim 2 wherein the polyphenol is a flavanoid, flavonoid, flavonoid derivative, flavolignan, polyphenolic rhizome, or a mixture thereof.

4. The composition of claim 3 wherein the flavanoid is catechin, a catechin derivative, or a mixture thereof.

5. The composition of claim 4 wherein the catechin derivative is an epictechin, a gallocatechin, an epigallocatechin, an epicatechin gallate, or an epigallocatechin gallate.

6. The composition of claim 4 wherein the catechin, catechin derivative, or mixture thereof, is derived from green tea or comprised in a green tea extract.

7. The composition of claim 4 wherein the catechin, catechin derivative, or mixture thereof, derived from cacoa or comprised in a cacoa extract.

8. The composition of claim 3 wherein the flavanoid is anthocyanin or oligomeric proanthocyanidin.

9. The composition of claim 3 wherein the flavonoid is quercetin, kaempferol, myricetin, rutin, or baicalein.

10. The composition of claim 3 wherein the flavanoid derivative is quercetin chalcone or Ginkgo flavone glycoside.

11. The composition of claim 3 wherein the flavolignan is silybin, silydianin, or silychristin.

12. The composition of claim 3 wherein the polyphenolic rhizome is curcumin.

13. The composition of claim 1 wherein the phytochemical compound is a carotenoid.

14. The composition of claim 13 wherein the carotenoid is lycopene.

15. The composition of claim 13 wherein the carotenoid is astaxanthin.

16. The composition of claim 13 wherein the carotenoid is lutein, alpha-carotene, beta-carotene, canthaxanthin, or zeaxanthin.

17. The composition of claim 1 wherein the phytochemical compound is a mixture of a flavanoid and a carotenoid.

18. The composition of claim 1 wherein the peptide copper complex is alanyl-histidyl-lysine:copper(II).

19. The composition of claim 1 wherein the peptide copper complex is valyl-histidyl-lysine:copper(II).

20. The composition of claim 1 wherein the peptide copper complex is glycyl-histidyl-lysine:copper(II).

21. The composition of claim 1 wherein the peptide copper complex is L-alanyl-L-histidyl-L-lysine:copper(II), L-valyl-L-histidyl-L-lysine L-lysine:copper(II).

22. The composition of claim 1 wherein the peptide copper complex is [glycyl-histidyl-lysine-R]:copper(II), wherein R is an alkyl moiety containing from one to eighteen carbon atoms, an aryl moiety containing from six to twelve carbon atoms, an alkoxy moiety containing from one to twelve carbon atoms, or an aryloxy moiety containing from six to twelve carbon atoms

23. The composition of claim 1 wherein the molar ratio of peptide to copper in the peptide copper complex ranges from about 1:1 to about 3:1.

24. The composition of claim 1 wherein the molar ratio of peptide to copper in the peptide copper complex ranges from about 1:1 to about 2:1.

25. The composition of claim 1 wherein the peptide copper complex is present at a concentration ranging from about 0.01% to about 10% by weight of the composition.

26. The composition of claim 1 wherein the peptide copper complex is present at a concentration ranging from about 0.025% to about 1% by weight of the composition.

27. The composition of claim 1 wherein the peptide copper complex is present at a concentration ranging from about 0.05% to about 0.5% by weight of the composition.

28. The composition of claim 1 wherein the phytochemical compound is present at a concentration ranging from about 0.001% to about 10% by weight of the composition.

29. The composition of claim 1 wherein the phytochemical compound and the peptide copper complex is encapsulated in a liposome or microsponge adapted to aid in the delivery of the peptide copper complex, or to enhance the stability of the composition.

30. The composition of claim 1 wherein the phytochemical compound and the peptide copper complex are formulated in an instrument adapted to deliver the compounds via iontophoresis.

31. The composition of claim 1, further comprising an inert and physiologically-acceptable carrier, thereby, suitable for oral or parenteral administration.

32. The composition of claim 31 wherein the inert and physiologically-acceptable carrier is sterile water, physiological saline, bacteriostatic saline, or phosphate-buffered saline.

33. The composition of claim 1, further comprising an inert and physiologically-acceptable diluent, thereby, suitable for topical administration to the skin.

34. The composition of claim 33 wherein the inert and physiologically-acceptable diluent is saline, sterile water, a petrolatum based cream, a pharmaceutically acceptable gel, a short chain alcohol, or a short chain glycol.

35. The composition of claim 34, further comprising a sunscreen agent.

36. The composition of claim 34, further comprising a skin conditioning agent.

37. The composition of claim 34, further comprising a skin protectant.

38. The composition of claim 34, further comprising an emollient.

39. The composition of claim 34, further comprising a humectant.

40. The composition of claim 34, further comprising a fatty alcohol, a fatty acid, an organic base, an inorganic base, a preserving agent, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, or a mixture thereof.

41. The composition of claim 34, further comprising an emulsifying agent, a surfactant, a thickening agent, an excipient, or a mixture thereof.

42. The composition of claim 34 wherein the composition is in the form of a liquid, cream, gel, fluid cream, lotion, or oil.

43. A method for enhancing or restoring the resistance of a mammal to oxidative or inflammatory damage caused by the release of reactive oxygen species, comprising orally, parenterally, or topically administering to the mammal a therapeutically effective amount of the composition of claim 1.

44. A method for accelerating the healing of wounds in a mammal, comprising orally, parenterally, or topically administering to the mammal a therapeutically effective amount of the composition of claim 1.

45. A method for cosmetically treating mammalian skin, comprising contacting the skin in need thereof with an effective amount of the composition of claim 42.

46. The method of claim 45 wherein the cosmetic treatment of the mammalian skin is smoothening the skin, reducing hyperpigmentation of the skin, reducing wrinkles and fine lines in the skin, reducing evidence of photodamage of the skin, or reducing the signs of aging in the skin.

47. A method for stimulating hair growth, preventing hair loss, or treating hair loss, comprising orally, parenterally, or topically administering to a mammal an effective amount of the composition of claim 1.
